(19) United States
(12) Patent Application Publication
(10) Pub. No.: US 2019/0142991 A1
(43) Pub. Date: May 16, 2019

(54) OPTIMIZED COMPOSITION FOR INTERFACE DRESSING

(71) Applicant: URGO RECHERCHE INNOVATION ET DEVELOPPEMENT, Chenove (FR)

(72) Inventors: Stephane AUGUSTE, Ruffey Les Echirey (FR); Anne-Sophie DANEROL, Dijon (FR); Nadege DESMAISON, Tart Le Haut (FR); Annick GENOT, Dijon (FR); Francoise JOLY, Couternon (FR); Aurelie LABORDE, Marsannay-La-Cote (FR); Didier GALLAND, Navilly (FR); Christelle GUILLAMAUD, Chenove (FR)

(21) Appl. No.: 16/098,391

(22) PCT Filed: May 4, 2017
(86) PCT No.: PCT/FR2017/051067

§ 371 Date: Nov. 1, 2018

(30) Foreign Application Priority Data
May 4, 2016 (FR) 1654051

(51) Int. Cl. A61L 15/24; A61L 15/42; A61L 15/44; A61L 15/20; C08L 53/02; A61L 15/22

(52) U.S. Cl. CPC A61L 15/24; A61L 15/425; A61L 15/42; A61L 15/44; A61L 15/20; C08L 2205/025; A61L 15/225; A61L 2300/404; A61L 2300/402; C08L 2203/02; C08L 53/025

(57) ABSTRACT

The present invention relates to a composition which has a low affinity with respect to latex gloves, comprising a hydrophobic matrix, characterized in that said hydrophobic matrix comprises:
for 100 parts by weight of a mixture P of 2 styrene-saturated olefin-styrene triblock copolymers, a first which has a viscosity of between 0.01 and 1 Pa·s as measured in a 5% (weight/weight) solution in toluene and a second which has a viscosity of between 0.01 and 0.5 Pa·s measured in a 15% (weight/weight) solution in toluene;
from 300 to 1000 parts by weight of a plasticizer H, preferably a plasticizing oil; and
from 90 to 600 parts by weight of petroleum jelly V;
   it also being specified that:
the total amount, represented by P+H+V, of the mixture of elastomers, of plasticizer and of petroleum jelly is between 490 and 1700 parts by weight;
the ratio between the total amount of the mixture of elastomers, of plasticizer and of petroleum jelly and the amount of petroleum jelly, represented by P+H+V/V, is less than 11;
   and said mixture of 2 copolymers comprises at least 20% by weight of the first copolymer.

The present invention also relates to an interface dressing with reinforcement, with a support or which is self-supported, which dressing comprises such a composition.

The present invention relates to a novel composition based on copolymers which are solely ABA triblock copolymers, comprising two styrene thermoplastic end blocks A and an elastomer central sequence B which is a saturated olefin, that can be used in particular for producing an interface dressing with reinforcement, with a support or which is self-supported, which has a low affinity with respect to latex surgical gloves.

The present invention also relates to an optimized interface dressing which exhibits facilitated handleability since it “does not adhere” to latex surgical gloves, by virtue of the use of this novel composition.

The treatment of wounds using dressings intended to be brought into contact with the wound while ensuring an interface between said wound and an absorbent compress that is placed on the dressing in order absorb the wound exudates has been known for a long time. Such dressings are usually referred to by the expression “interface dressings”.

The dressing sold since 2000 by the company URGO Laboratories under the name URGOTUL® is an illustrative example of such dressings.

This product consists of a reinforcement made of an open-mesh fabric, the threads of which are coated with a cohesive gel, so as to leave the meshes essentially unblocked.

This gel is formed from a composition consisting of a hydrophobic elastomeric matrix based on ABA (styrene-saturated olefin-styrene) triblock copolymers, which matrix is highly plasticized and contains, in a dispersion, a small amount of hydrophilic particles of a hydrocolloid.

This dressing and the composition thereof are described in example 1 of patent application WO 00 16725.

The qualitative and quantitative composition of the elastomeric matrix of this dressing confers on it notable properties with regard to the promoting of the healing process and in particular of fibroblast proliferation.

The URGOTUL® product nevertheless has the drawback, in the case where it is desired to apply it to wounds that are difficult to cover, for example owing to their location, of lacking conformability because of the rigidity of its reinforcement.

To solve this problem, self-supported interface dressings have been described in patent application FR 2 936 158. The solutions proposed in this patent application make it possible to obtain products which exhibit both a good elasticity and sufficient rigidity and also sufficient cohesion to be able to handle them.

The solution proposed in FR 2 936 158 does not use triblock polymers with a saturated central sequence, but compounds which are in addition to or different than those used in URGOTUL®.

However, for the purposes of economic cost effectiveness, it would be preferable to be able to produce a self-supported interface dressing with compounds which are identical to or of the same nature as those used for the production of the URGOTUL® dressing and variations thereof.

This would also have a not insignificant advantage in the case where it is desired to incorporate into this composition active compounds, that is to say compounds which have an action on the healing process or the treatment of the wound, for instance antibacterial agents, such as silver salts, or MMP (matrix metalloprotease) inhibitors such as the potassium salt of sucrose octasulfate.

Indeed, the incorporation of active agents into this type of composition is always difficult and complex, it being possible for each compound of the composition to interact with the others or to modify the rheological and physicochemical properties of the composition, or even to affect the stability or the solubility of the active agent.

If compounds which are identical to or of the same nature as those already used for the production of the URGOTUL® dressing are used, the chances of developing new compositions having the desired advantages are thus optimized.

Finally, the use of compounds which are identical to or of the same nature as those used for the production of the URGOTUL® dressing would make it possible to prevent any degradation or modification, in the new compositions desired, of the notable properties of the URGOTUL® product with regard to fibroblast proliferation and to the healing process.

Patent application FR 2 936 159 describes another variant of interface dressing in which the latter comprises a support, covered on at least its face which comes into contact with the wound, with a composition based on a hydrophobic elastomeric matrix, the assembly thus formed being perforated so as to obtain through-holes. The compositions used according to this document are similar to those described in application FR 2 936 158 and have the same properties and drawbacks.

Patent applications EP 2 524705 and EP 2 524706 describe interface dressings with reinforcement, which are produced from copolymers which are solely ABA triblock copolymers, comprising two styrene thermoplastic end blocks A and an elastomer central sequence B which is a saturated olefin.

However, the interface dressings sold at the current time or proposed in these prior documents suffer from a high affinity with respect to latex surgical gloves, which is often described as “adhesion” to latex gloves.

Thus, although the URGOTUL® product has been sold for 15 years, it has never been possible to solve this problem of affinity or adhesion to latex gloves.

There are many consequences of this phenomenon.

In the case of a reinforcement-free self-supported dressing, this affinity with respect to latex surgical gloves results in the fact that the interface dressing “sticks” to said gloves, thus preventing the dressing from being handled and from being correctly and rapidly applied to the wound. The care staff are confronted with a significant handicap for rapid and efficient use of the interface dressing.

In the case of a dressing with reinforcement, such as the URGOTUL® product, the attachment of the composition deposited on the reinforcement is weak and said composition can even be transferred onto the latex glove, which then makes the product unusable.

In order to avoid this problem, it is recommended, in the information sheet for the URGOTUL® product, to wet the gloves with physiological saline in order to facilitate the handling thereof. The contact between the hydrocolloid particles and the physiological saline in fact leads to a beginning of gelling of these particles, which decreases the phenomenon of “adhesion” to the gloves.

Another possible solution for avoiding this problem of adhesion to the gloves is to handle the product only with forceps, but once again its use becomes complex, in particular for certain wound locations.

In order to obtain an optimal interface dressing, which is easy and economical to produce, it would therefore be desirable to have a composition for a dressing, which, with compounds identical to or of the same nature as those used in URGOTUL®, “does not adhere” to latex gloves, this being both for an interface dressing with reinforcement and for a dressing with support or which is self-supported.

The cause of this phenomenon of affinity with respect to latex gloves has not for the moment been determined, but is thought to be due, a priori, to the composition of the hydrophobic elastomeric matrix.

The analysis of the affinity of elastomeric matrices with respect to latex gloves is very complex.

This is because this “adhesion” can vary as a function of the nature of the latex gloves and of their possible surface treatment. In addition, it is very difficult to develop a test which mimics this phenomenon of adhesion of the composition to a glove pulled onto a hand. Indeed, when a glove is pulled onto a hand, its surface is modified as a function of the morphology of the hand and of the fingers, which impose particular radii of curvature and apply a variable tension to the glove, which results in different surface contacts with the composition. Furthermore, even if the “adhesion” phenomenon is striking, the forces to be measured are very weak. Finally, the elasticity properties of the compositions go against the use of commonly used techniques for measuring adhesive capacity.

As a result, it has not been possible, up until now, to carry out adhesion measurements that are sufficiently accurate and reliable to obtain significant differences making it possible to distinguish a composition which “adheres” from a composition which “does not adhere” to latex gloves.

This doubtless explains, at least partly, why no solution has been proposed for approximately 15 years, in order to remedy this phenomenon of adhesion of interface dressings to latex gloves.

After numerous failures, the inventors have succeeded in developing a reliable and reproducible method for evaluating this affinity by carrying out a measurement of adhesive power with a 90 degree peel using specific mock-ups of compositions which make it possible to obtain discriminating results with a reference glove on which “the adhesion” of the URGOTUL® product is particularly marked.

By virtue of the development of this test, the inventors have finally been able to determine the parameters that a composition must have in order to have an affinity with respect to latex gloves that is sufficiently low to allow them to be easily handled and to consider that this composition “does not adhere” to latex gloves.

Thus, the present invention has made it possible to solve, for the first time, the technical problem consisting of the development of a specific composition based on copolymers which are solely ABA triblock copolymers, comprising two styrene thermoplastic end blocks A and an elastomer central sequence B which is a saturated olefin, which composition consists of the compounds normally used in the URGOTUL® dressing and which, by virtue of its low affinity with respect to latex surgical gloves, is in particular usable both for producing a self-supported interface dressing and for producing a dressing with reinforcement or with a support.

More specifically, it has been discovered, and this constitutes the foundation of the present invention, that compositions of which the hydrophobic matrix comprises a mixture of 2 styrene-saturated olefin-styrene specific triblock copolymers (elastomers), in which the first copolymer has a viscosity of between 0.01 and 1 Pa·s as measured in a 5% (weight/weight) solution in toluene and the second copolymer has a viscosity of between 0.01 and 0.5 Pa·s as measured in a 15% (weight/weight) solution in toluene, said mixture of copolymers being present within the composition in a predetermined weight amount and in relative proportions with the other components of the matrix (plasticizer and petroleum jelly) specifically chosen, make it possible to produce dressings which have a sufficiently low affinity with respect to latex surgical gloves to prevent them from “adhering” thereto.

Thus, according to a first aspect, a subject of the present invention is a composition, which is in particular of use for the production of dressings, which has a low affinity with respect to latex gloves, said composition comprising a hydrophobic elastomeric matrix, characterized in that said hydrophobic matrix comprises:
for 100 parts by weight of a mixture P of 2 styrene-saturated olefin-styrene specific triblock copolymers, a first which has a viscosity of between 0.01 and 1 Pa·s as measured in a 5% (weight/weight) solution in toluene and a second which has a viscosity of between 0.01 and 0.5 Pa·s as measured in a 15% (weight/weight) solution in toluene;
from 300 to 1000 parts by weight of a plasticizer H, preferably a plasticizing oil; and
from 90 to 600 parts by weight of petroleum jelly V;
   it also being specified that:
the total amount, represented by P+H+V, of the mixture of elastomers, of plasticizer and of petroleum jelly is between 490 and 1700 parts by weight;
the ratio between the total amount of the mixture of elastomers, of plasticizer and of petroleum jelly and the amount of petroleum jelly, represented by P+H+V/V, is less than 11;
   and said mixture of 2 copolymers comprises at least 20% by weight of the first copolymer.

According to one particular feature, the abovementioned hydrophobic matrix also comprises hydrocolloid particles in an amount of less than or equal to 25% by weight, relative to the total weight of said hydrophobic matrix.

According to a second aspect, a subject of the present invention is an interface dressing with reinforcement, with a support or which is self-supported, which dressing comprises such a composition.

The composition according to the invention which has a low affinity with respect to latex surgical gloves comprises a combination of 2 copolymers, which are solely ABA triblock copolymers, belonging respectively to predetermined categories.

The sequenced copolymers used in the context of the invention are ABA triblock copolymers comprising two styrene thermoplastic end blocks A and an elastomer central sequence B which is a saturated olefin. The sequences B of saturated olefins are, for example, ethylene-butylene, ethylene-propylene or ethylene-ethylene-propylene sequences.

In the interest of simplicity, in the present description, the polymeric blocks constituting the abovementioned copolymers are denoted by the nature of their repeating units. Thus, the expression “styrene block A” or “styrene sequence A” denotes a poly(styrene) sequence and the expression “saturated olefin block” or “saturated olefin sequence” denotes a poly(saturated olefin) sequence.

The copolymers which are solely triblocks with a saturated central sequence are well known to those skilled in the art and are for example sold:
by the company KRATON POLYMERS under the name KRATON® G, and in particular the grades KRATON® G1651, KRATON® G1654, KRATON® G1657, KRATON® G1652 or KRATON® G1650 and by the company KURARAY under the names SEPTON® and in particular the grades 8006 or 8004 for the poly(styrene-ethylene-butylene-styrene) (abbreviated to SEBS) sequenced copolymers;
by the company KURARAY under the name SEPTON® for the poly(styrene-ethylene-propylene-styrene) (abbreviated to SEPS) sequenced copolymers and in particular the grades 2005, 2006 or 2063 and for the poly(styrene-ethylene-ethylene-propylene-styrene) (abbreviated to SEEPS) sequenced polymers and in particular the grades 4033, 4044, 4055, 4077 or 4099.

In the context of the present invention, SEBS, SEPS or SEEPS triblock copolymers having a styrene content of between 25% and 45% by weight relative to the weight of said SEBS, SEPS or SEEPS copolymer are preferred.

Among the copolymers which have a viscosity of between 0.01 and 1 Pa·second measured in a 5% (weight/weight) solution in toluene, mention may be made of the copolymers sold by the company KRATON under the grades KRATON® G1651 and KRATON® G1654 and the copolymers sold by the company KURARAY under the grades SEPTON® 2005, 2006, 8006, 4044, 4055, 4077 or 4099.

Among the copolymers which have a viscosity of between 0.01 and 0.5 Pa·second measured in a 15% (weight/weight) solution in toluene, mention may be made of the copolymers sold by the company KRATON under the grades KRATON® G1650, KRATON® G1657 and KRATON® G1652 and the copolymers sold by the company KURARAY under the grades SEPTON® 2063 or 4033.

These viscosities are measured at 30° C. using a Brookfield model LTV viscometer in a 5% or 15% weight/weight solution in toluene as a function of the molecular weight of the copolymer.

In general, this mixture of 2 copolymers will comprise at least 20% by weight of the copolymer which has a viscosity of between 0.01 and 1 Pa·second measured in a 5% weight/weight solution in toluene.

Preferably, this proportion will be greater than 30%. In particular, for the production of self-supported interface dressings, mixtures which comprise at least 50% and preferably at least 70% of copolymer which has a viscosity of between 0.01 and 1 Pa·s will preferably be used in order to obtain not only a low affinity with respect to latex gloves, but also the best cohesion and elasticity properties.

In general, the amount of the mixture of these 2 copolymers in the final composition may be between 5% and 20% by weight, preferably between 5% and 15% and more particularly between 7% and 10% by weight, relative to the total weight of the composition.

Contrary to what might have been expected, it has been observed that the mixture of these copolymers, in the proportions defined above, results in homogeneous compositions which have good cohesion properties despite the use of 2 triblock copolymers with such different viscosities.

In the context of the present invention, preference will most particularly be given to the use of two SEBS sequenced copolymers, and in particular the combination of the copolymers KRATON® G1654 and KRATON® G1650 in which the KRATON® G1654/KRATON® G1650 weight percent distribution is between 50/50 and 70/30, in a total amount which preferably represents between 5% and 15% and preferably between 7% and 10% by weight, relative to the total weight of the composition.

In order to produce interface dressings, the mixture of these 2 copolymers is combined with one (or more) plasticizing compound(s) and with petroleum jelly so as to form a hydrophobic matrix.

In order to obtain compositions which have a low affinity with respect to latex gloves, this hydrophobic matrix necessarily comprises these three types of constituents (mixture of copolymers, plasticizer and petroleum jelly), in specific proportions.

In the context of the invention, the petroleum jelly is a petroleum jelly in accordance with the commercially available French Pharmacopeia.

The plasticizers which can be used are well known and are intended to improve the stretching, flexibility, extrudability or processing properties of the copolymers. To this effect, use may be made of one or more plasticizers if necessary.

In general, as plasticizers, preference will be given to liquid compounds which are compatible with the saturated olefin central sequence of the abovementioned sequenced copolymers, and more particularly the compounds which have a dropping point of less than or equal to 35° C.

Among the plasticizing compounds which can be used to this effect, mention will in particular be made of plasticizing mineral oils.

Alternatively, it is also possible to use synthesis products based on liquid mixtures of saturated hydrocarbons, for instance the products sold by the company TOTAL under the name GEMSEAL® and in particular the product GEMSEAL® 60 which is an isoparaffin mixture derived from a totally hydrogenated petroleum fraction.

In the context of the present invention, use will preferably be made of plasticizing oils and in particular mineral oils formed from compounds of paraffinic or naphthenic nature, or mixtures thereof, in variable proportions.

Particularly preferred plasticizing mineral oils are formed from mixtures of compounds of paraffinic and naphthenic nature, and in particular such mixtures in which the proportion of compounds of paraffinic nature is predominant.

Among the plasticizing oils that are particularly suitable, mention may be made of the products sold by the company SHELL under the names ONDINA® and in particular ONDINA® 919 or the oil sold by the company PETRO CANADA under the reference PURETOL® 9D.

As previously indicated, the amounts of plasticizer and of petroleum jelly must be chosen as a function of the total amount of elastomer constituted by the abovementioned mixture of copolymers.

On the basis of the test that will subsequently be described, it is considered that a composition which has an adhesive power of less than or equal to 7 cN/cm measured on a glove sold by the company VWR under the reference 112-1567 “does not adhere” to latex gloves.

In order to obtain this result, it has been determined that the hydrophobic matrix must comprise:
for 100 parts by weight of a mixture P of 2 specific styrene-saturated olefin-styrene triblock copolymers, a first which has a viscosity of between 0.01 and 1 Pa·s as measured in a 5% (weight/weight) solution in toluene and a second which has a viscosity of between 0.01 and 0.5 Pa·s measured in a 15% (weight/weight) solution in toluene;
from 300 to 1000 parts by weight of a plasticizer H, preferably a plasticizing oil; and
from 90 to 600 parts by weight of petroleum jelly V;
   it also being specified that:
the total amount, represented by P+H+V, of the mixture of copolymers, of plasticizer and of petroleum jelly is between 490 and 1700 parts by weight;
the ratio between the total amount of the mixture of copolymers, of plasticizer and of petroleum jelly and the amount of petroleum jelly, represented by P+H+V/V, is less than 11;
   and said mixture of 2 copolymers comprises at least 20% by weight of the first copolymer.

In the subsequent text, and in the interest of simplicity:
the expression “the total amount, represented by P+H+V, of the mixture of copolymers, of plasticizer and of petroleum jelly is between X and Y parts by weight” will sometimes be replaced by the equation X<P+H+V<Y; and
the expression “the ratio between the total amount of the mixture of copolymers, of plasticizer and of petroleum jelly and the amount of petroleum jelly, represented by P+H+V/V, is less than Z” will sometimes be replaced by the equation P+H+V/V<Z;
   it being specified that X, Y and Z denote integers.

For the production of self-supported interface dressings which have the best balance between elasticity and cohesion, preference will be given to a composition of which the hydrophobic matrix comprises:
for 100 parts by weight of a mixture P of 2 specific styrene-saturated olefin-styrene triblock copolymers, a first which has a viscosity of between 0.01 and 1 Pa·s as measured in a 5% (weight/weight) solution in toluene and a second which has a viscosity of between 0.01 and 0.5 Pa·s measured in a 15% (weight/weight) solution in toluene;
from 500 to 1000 parts by weight of a plasticizer H, preferably a plasticizing oil; and
from 100 to 400 parts by weight of petroleum jelly V;
   it also being specified that:

the following equations are satisfied:

700<P+H+V<1200 and

(P+H+V)/V<8

and said mixture of 2 copolymers comprises at least 30% by weight of the first copolymer.

According to one currently preferred embodiment of the present invention, which makes it possible to obtain the most effective interface dressings and the composition which has the lowest affinity with respect to latex gloves, the hydrophobic matrix comprises:
for 100 parts by weight of a mixture P of 2 specific styrene-saturated olefin-styrene triblock copolymers, a first which has a viscosity of between 0.01 and 1 Pa·s as measured in a 5% (weight/weight) solution in toluene and a second which has a viscosity of between 0.01 and 0.5 Pa·s measured in a 15% (weight/weight) solution in toluene;
from 650 to 800 parts by weight of a plasticizer H, preferably a plasticizing oil; and
from 150 to 200 parts by weight of petroleum jelly V;
   it also being specified that:

the following equations are satisfied:

900<P+H+V<1100 and

(P+H+V)/V<6

and said mixture of 2 copolymers comprises at least 50% by weight of the first copolymer.

On the basis of the test developed by the inventors, such compositions have an adhesive power of less than or equal to 3 cN/cm, measured on a glove sold by the company VWR under the reference 112-1567, and are particularly suitable for producing a self-supported interface dressing which is easy to handle with latex surgical gloves.

These compositions are also of use for preparing dressings with reinforcement or with a support.

The hydrophobic matrix which has just been described constitutes the essential element of the compositions making it possible to obtain a composition and a dressing which have a low affinity with respect to latex gloves in accordance with the invention.

Such compositions can nevertheless comprise additional compounds and in particular compounds chosen from antioxidants, hydrocolloids, and active agents or adjuvants commonly used in the wound treatment field.

The term “antioxidants” is intended to denote herein the compounds commonly used by those skilled in the art to provide the stability of the compounds which are part of the formulation of the compositions, in particular with respect to oxygen, heat, ozone or ultraviolet radiation.

As examples of suitable antioxidants, mention may in particular be made of phenolic antioxidants, such as in particular the products sold by the company BASF under the names IRGANOX® 1010, IRGANOX® 565 and IRGANOX® 1076.

In general, these antioxidants may be used alone or in combination in an amount of about from 0.05% to 1% by weight, preferably from 0.05% to 0.2% by weight, relative to the total weight of the composition.

In the context of the present invention, the use of the IRGANOX® 1010 product in an amount of between 0.05% and 0.2% by weight, relative to the total weight of the composition, will be preferred.

According to another embodiment of the invention that is particularly preferred in the context of the production of interface dressings which are self-supported, with a support or with reinforcement, for wound healing, the compositions according to the invention comprise hydrophilic particles of a hydrocolloid (or hydrocolloid particles).

These particles in fact allow the painless removal of an interface dressing and the maintaining of a wet environment at the level of the wound in order to promote healing.

To this effect, a small amount of hydrophilic particles of a hydrocolloid is thus either placed at the surface of the hydrophobic matrix or, preferably, homogeneously dispersed within the composition.

The term “hydrocolloid” or “hydrocolloid particles” is intended to denote herein any compound normally used by those skilled in the art for its ability to absorb aqueous liquids such as water, physiological saline or wound exudates.

As suitable hydrocolloids, mention may for example be made of pectin, alginates, natural plant gums, such as in particular karaya gum, cellulose derivatives, such as carboxymethylcelluloses and alkali metal, such as sodium or calcium, salts thereof, and also synthetic polymers based on acrylic acid salts, known under the name “superabsorbents”, such as for example the products sold by the company BASF under the name LUQUASORB® 1003 or by the company CIBA Specialty Chemicals under the name SALCARE® SC91 and also the mixtures of these compounds.

Some of these superabsorbants described as “microcolloids” since they have a particle size of less than 10 micrometers may of course also be used.

The preferred hydrocolloids in the context of the present invention are alkali metal salts of carboxymethylcellulose, and in particular sodium carboxymethylcellulose (CMC).

The size of the hydrocolloid particles is generally between 50 and 100 microns, advantageously about 80 microns.

In general, the amount of hydrocolloid particles incorporated into the composition according to the invention will advantageously be less than or equal to 25% by weight, relative to the total weight of said hydrophobic matrix.

If the hydrocolloid particles are placed at the surface of the hydrophobic matrix, the amount of said particles will preferably be about from 1% to 10% and more particularly from 2% to 5% by weight, relative to the total weight of said hydrophobic matrix.

The hydrocolloid particles are preferably homogeneously dispersed within the composition.

The amount of hydrocolloid particles dispersed in the composition will in this case advantageously be about from 2% to 20% by weight, preferably from 5% to 18% by weight, more preferably from 10% to 15% by weight, relative to the total weight of the hydrophobic matrix.

The choice of an amount of hydrocolloid particles included in these value ranges is important for the production of an interface dressing, and in particular a breathable self-supported interface dressing, in order to prevent gelling of the composition from causing closure of the through-holes during the absorption of exudates.

According to another embodiment, the compositions according to the invention comprise one or more adjuvants and/or active agents commonly used in the wound treatment field or in the pharmacological field.

The composition can thus contain active agents which have a favorable role in wound treatment and which are in particular capable of inducing or accelerating healing during the wound detersion and/or granulation phase.

The presence of hydrocolloids within the composition will promote the release of these active agents.

These active agents may be used in an amount of about from 0.01% to 20% by weight, preferably from 1% to 15% by weight, and more preferably from 2% to 10% by weight, relative to the total weight of the composition.

Among the active agents capable of being used in the context of the invention, mention will for example be made of bactericidal or bacteriostatic agents, agents which promote healing, painkillers or local anesthetics and also anti-inflammatory agents.

By way of adjuvants which can be used in the compositions according to the invention, mention may be made of compounds known to promote the release of active agents, such as for example the products Montanox® 80 or Sepinov® EMT 10 which are commonly used in the URGOTUL® products which incorporate active agents.

These adjuvants may be used in an amount of about from 1% to 15% relative to the total weight of the composition.

Of course, the particular embodiments which have just been described can be implemented separately or according to any one of the combinations thereof.

The compositions according to the invention make it possible in particular to produce interface dressings which are self-supported, with a reinforcement or with a support.

In the context of the production of an interface dressing, the use of a composition which comprises compounds (copolymers, mineral oil, petroleum jelly, antioxidant and hydrocolloids) identical to, or even of the same nature as, those used in the URGOTUL® product will be preferred.

In order to produce a dressing, the compositions according to the invention will be formed into a thin layer, with through-holes, preferably arranged in a manner distributed in said layer.

The through-holes can be produced by perforation or punching of a composition preformed into a thin layer, alone or combined with a temporary support or with a protective film normally used for the production of a dressing, or else by a screened coating on a temporary support.

Alternatively, the dressings in accordance with the invention can be produced by hot casting of a composition as previously described on a plate engraved with the pattern used for forming the through-holes, followed by cooling and demolding.

In general, the dressings in accordance with the invention will have a thickness of between 0.4 mm and 2 mm, preferably between 0.5 mm and 1 mm, more preferably of about 0.6 mm.

The through-holes may be of any geometry and will have, for example, a circular, rectangular, trapezoidal or square cross section.

Their surface area will generally be between 0.25 and 5 mm².

These holes will in particular have a mean diameter of between 0.5 and 2 mm, preferably of about 1 mm, when their cross section is circular.

These holes will be distributed, preferably uniformly, with a density such that the total surface area of the holes represents between 20% and 70%, and preferably between 30% and 50%, of the total surface area of the dressing.

According to one preferred embodiment, the self-supported interface dressing according to the invention is in the form of a breathable net (or grid), preferably of square mesh, having:
a thickness of the net of between 0.5 and 2 mm;
a “yarn width” (width of the space between two consecutive holes) of between 1 and 10 mm, and preferably between 1 and 5 mm;
a grammage of between 200 and 1700 g/m², and preferably of between 300 and 800 g/m².

According to one particularly preferred embodiment of the invention, such a dressing will be in the form of a square-mesh breathable net having:
a thickness of the net of approximately 600 microns;
a yarn width (or mesh size) of about 2 mm;
a grammage of about 450 g/m².

For the production of such self-supported dressings, reference will be made, for further details, to patent application FR 2 936 158.

According to one currently preferred embodiment, the present application aims to cover a self-supported interface dressing comprising a thin layer having through-holes in order to allow exudates to pass, characterized in that the composition of the hydrophobic matrix comprises:
for 100 parts by weight of a mixture P of 2 specific styrene-saturated olefin-styrene triblock copolymers, a first which has a viscosity of between 0.01 and 1 Pa·s as measured in a 5% (weight/weight) solution in toluene and a second which has a viscosity of between 0.01 and 0.5 Pa·s measured in a 15% (weight/weight) solution in toluene;
from 650 to 800 parts by weight of a plasticizer H, preferably a plasticizing oil; and
from 150 to 200 parts by weight of petroleum jelly V;
   it also being specified that:
the following equations are satisfied:

900<P+H+V<1100 and

(P+H+V)/V<6

said mixture of 2 copolymers comprises at least 50% by weight of the first copolymer;

and hydrocolloid particles in an amount of between 10% and 20% by weight relative to the total weight of the hydrophobic matrix.

The techniques for producing an interface dressing with reinforcement or with a support are also well known to those skilled in the art and reference may, for example, be made to the methods described in patent applications WO 00 16725 and FR 2 936 159.

In order to protect the composition from the external environment, the interface dressing will preferably be covered on each of its faces with a temporary protective film that will be removed before use by the user.

In order to further facilitate the handling of the interface dressing, in particular if it is self-supported, these two temporary protectors may be replaced with a single protector as described in patent application WO 2008/145884, the particular structure of which facilitates the application of the dressing to the wound.

Of course, the compositions according to the invention may also be used in the production of any product, and in particular any medical device which is capable, when it is used, of being handled with latex gloves.

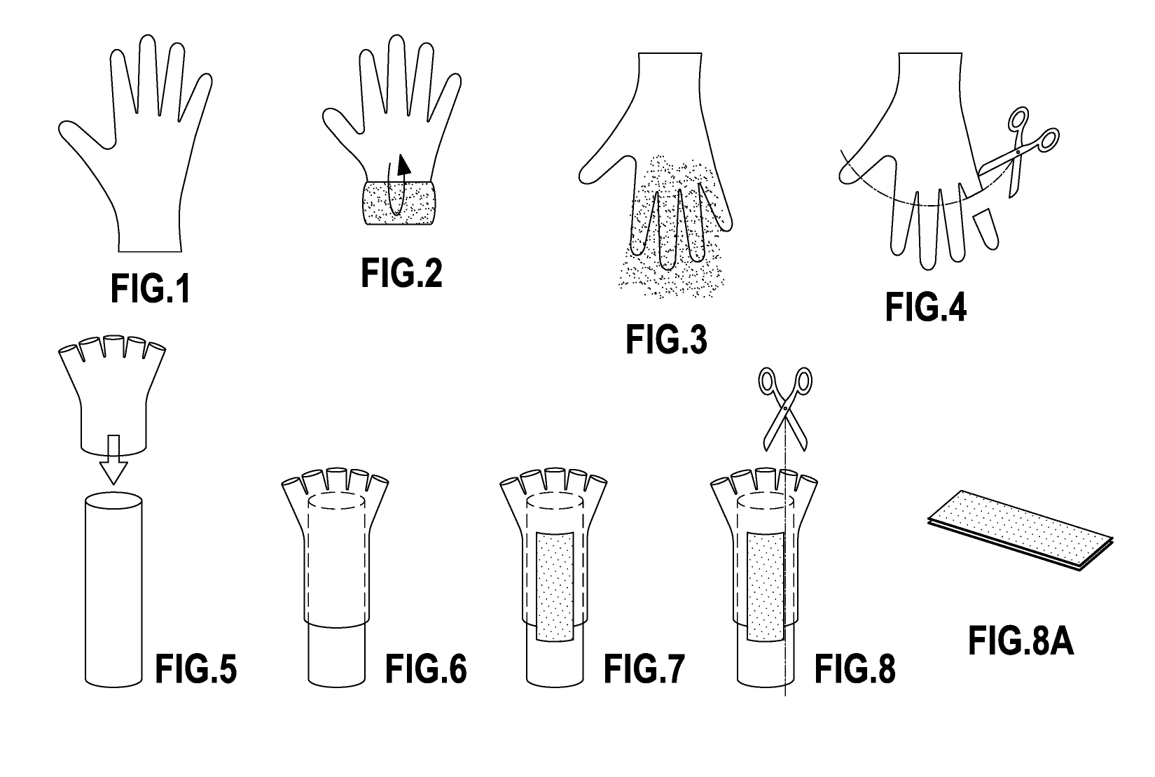

The invention will now be illustrated by the following examples and tests, with reference to the appended drawings in which FIGS. 1 to 13 illustrate the various steps of the test developed by the inventors for measuring the adhesion to latex gloves.

Preparation of the Compositions

The compositions of examples 1 to 7 were produced using the following constituents in the proportions, expressed in weight percent, mentioned in table 1.
Elastomer: sequenced copolymer of poly(styrene-ethylene-butylene-styrene) (abbreviated to SEBS):
KRATON® G1651 viscosity at 5% (weight/weight) in toluene: 0.04 Pa·s
KRATON® G1654 viscosity at 5% (weight/weight) in toluene: 0.02 Pa·s
KRATON® G1652 viscosity at 15% (weight/weight) in toluene: 0.088 Pa·s
KRATON® G1650 viscosity at 15% (weight/weight) in toluene: 0.2 Pa·s
plasticizer: Ondina®919 mineral oil sold by the company SHELL
antioxidant: IRGANOX® 1010 sold by the company BASF
petroleum jelly: Codex® A petroleum jelly sold by the company AIGLON
hydrocolloid: sodium carboxymethylcellulose CMC BLANOSE® 7H4XF sold by the company ASHLAND.

Production of the Composition

The plasticizer, the hydrocolloid and the petroleum jelly were successively introduced, with stirring at a setpoint temperature of 90° C., into an IKA mixer and the mixture was blended until a homogeneous mixture was obtained.

After having brought the setpoint temperature to 140° C., the 2 copolymers and the antioxidant were introduced, with stirring, then the mixture was blended until a homogeneous mixture was obtained.

The mixture was then left to cool, then the mixer was emptied.

TABLE 1

| | Example 1 | Example 2 | Example 3 | Example 4 | Example 5 | Example 6 | Example 7 |
|---|---|---|---|---|---|---|---|
| Plasticizer | 74.95 | 75 | 75 | 61.5 | 68.48 | 68.48 | 68.48 |
| Petroleum jelly | 5 | | 4.95 | 15 | 8.02 | 8.02 | 8.02 |
| Antioxidant | 0.12 | 0.2 | 0.12 | 0.2 | 0.2 | 0.2 | 0.2 |
| Hydrocolloid | 15 | 10 | 15 | 15 | 15 | 15 | 15 |
| Kraton ® G1651 | 4.93 | | 2.96 | | | | |
| Kraton ® G1654 | | 7.8 | | 5.7 | 4.15 | 5.7 | 2.6 |
| Kraton ® G1650 | | 7 | | 2.6 | 4.15 | 2.6 | 5.7 |
| Kraton ® G1652 | | | 1.97 | | | | |

Demonstration of the Adhesion of the Compositions to Latex Gloves:

The conditions for carrying out the test and for producing the mock-ups used were the following.

Production of the Mock-Ups

The mock-ups were generally in the form of reinforced compositions in which a reinforcement is embedded in order to stiffen them.

The reinforcement used was a thermoset knit, made of polyester yarns, produced by the company MDB TEXINOV under the reference 555. The grammage thereof was 40 g/m². It had an extensibility, measured according to standard EN 13726-4, of 2.7 N/cm in the cross direction and 24 N/cm in the longitudinal direction.

2 sheets were thus produced using the compositions to be tested and this reinforcement was incorporated between the 2 sheets of composition, by applying a strong pressure using a hydraulic press on the assembly according to the following protocol.

The 2 plates of the press were preheated. A non-stick plastic film, for example a white silicone-treated/fluorinated polyester film, referenced 50 MD 07, sold by the company Siliconature, was deposited on the lower plate of the press; the silicone-treated/fluorinated face being placed opposite to the lower plate. 10 g of one of the compositions described in table 1 were spread over this face and the latter was covered with a silicone-treated polyester film 75 micrometers thick, the silicone-treated side being placed in contact with the composition. 2 blocks of 0.70 mm were placed between the 2 polyester films at the ends of the lower plate of the press and the assembly was subjected to a pressure of 200 bar and a temperature of about from 90 to 100° C.

A second sheet was produced in the same way.

A sample of the reinforcement previously defined on the apparent composition was applied on one of these sheets, after having removed the silicone-treated/fluorinated polyester.

Starting from the other of these sheets, after having removed the silicone-treated/fluorinated polyester, the face of this sheet, comprising the composition to be tested, was applied on the reinforcement surface-linked, to the other sheet, so as to thus obtain a silicone-treated polyester—composition—reinforcement—composition—silicone-treated polyester complex.

2 blocks of 1 mm and 0.2 mm, respectively, were placed between the 2 silicone-treated polyester films at the ends of the lower plate of the press and a pressure of 200 bar and a temperature of about from 90 to 100° C. were applied.

The mock-up was left to cool and its thickness was controlled with a micrometer so as to obtain a mock-up of which the thickness is about from 1 to 1.1 mm. It is in fact necessary to use such mock-ups of small thickness to carry out the test making it possible to measure the adhesion to latex gloves.

Measurement of the Adhesion to Latex Gloves

The adhesive power of the mock-ups produced according to the protocol previously described was measured using a conventional right-angle peel test. This peel test was carried out with an MTS dynamometer equipped with a 2-Newton sensor. This dynamometer makes it possible to measure the force required to detach the mock-up to be tested from a steel plate, covered with a sample of latex glove. This force is expressed in cN/cm.

The test was carried out at 23° C. and 50% relative humidity.

The procedure of the test was the following.

a. Preparation of the Steel Plate Covered with a Sample of Latex Glove

This preparation is illustrated by FIGS. 1 to 13 which describe the successive implementation steps.

A latex glove (size 8-9) referenced 112-1567 sold by the company VWR is used (FIG. 1).

The powdered latex glove is turned inside out (FIG. 2) with the user face on the inside. The glove thus turned inside out is shaken so as to remove the powder (FIG. 3) and the fingers and the thumb of the glove are cut off (FIG. 4). The glove, still turned inside out, is pulled onto a 1-liter cylinder 68 mm in diameter (FIGS. 5 and 6), the user face of the glove thus coming into contact with the cylinder. A strip of masking tape (sold by the company Plasto under the reference P 3650) 5 cm wide and 10 cm long is stuck to the glove without creating folds. The glove is cut along the edges of the masking tape so as to obtain a sample covered with masking tape on one of its faces (FIGS. 8 and 8A).

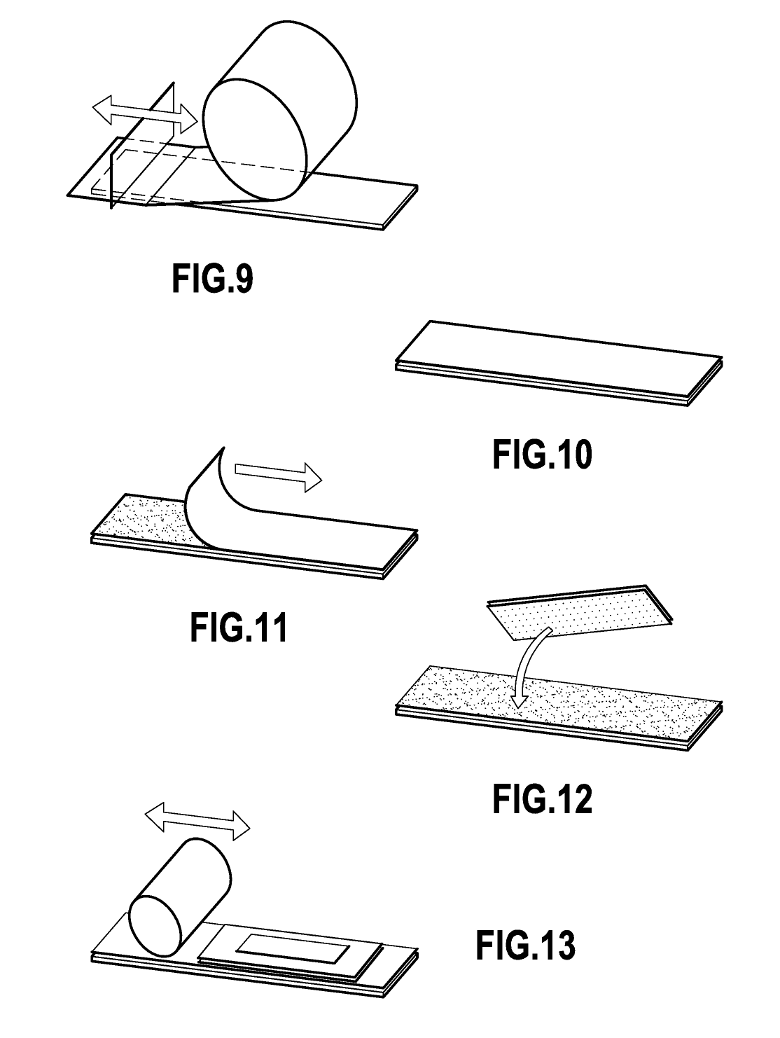

A double-sided adhesive sold by the company Plasto under the reference P753 is stuck on the steel plate used to carry out the peel test, while avoiding creating folds or bubbles (FIGS. 9 and 10). The protector is then detached from the double-sided adhesive (FIG. 11) and the masking-tape face of the sample of glove previously obtained is stuck onto the adhesive while avoiding folds and bubbles (FIG. 12). A test piece 1.5 cm wide and 10 cm long is cut from the mock-up to be tested and is deposited by centering it on the glove fixed to the steel plate as previously obtained and a 3 kg roller is passed back and forward across it twice at the speed of 200 mm/min (FIG. 13).

The 90 degree peel test was then carried out at the speed of 300 mm/min. The test was reproduced 5 times for each composition to be tested. The results obtained are the mean of these 5 measurements. The adhesive powers obtained, expressed with their standard deviation (i), are collated in table 2.

TABLE 2

| | Example 1 | Example 2 | Example 3 | Example 4 | Example 5 | Example 6 | Example 7 |
|---|---|---|---|---|---|---|---|
| Adhesive | 9.76 | 17.36 | 47.12 | 2.08 | 3.72 | 4.7 | 3.96 |
| power | i = 1.89 | i = 3.62 | i = 7.96 | i = 0.55 | i = 0.74 | i = 1.59 | i = 3.62 |

Examples 4 to 7 correspond to compositions according to the invention.

In addition, with the latex glove selected, it was noted that all the mock-ups corresponding to examples 4 to 7 according to the invention could be easily handled.

The compositions of examples 1 and 3 correspondent respectively to the formulations of examples 1 and 5 of patent application WO 00/16725. When the mock-ups which correspond to these 2 examples and that which corresponds to example 2 were handled with the latex glove selected, it was noted that all these mock-ups “adhered” to this latex glove, which made them very difficult to handle.

This clearly gave confirmation that the test carried out is representative of the affinity phenomenon. When the adhesive power, measured according to this test, is less than 7 cN/cm, the mock-ups “do not adhere” to latex gloves.

None of the compositions of comparative examples 1 to 3 meet this criterion of “non-adhesion”.

Moreover, a self-supported interface dressing was produced using the composition of example 4 which has the lowest adhesive power found, according to the following procedure.

The plasticizer and the hydrocolloid were successively introduced, with stirring at a setpoint temperature of 90° C., into a vertical mixer and the mixture was blended until a homogeneous mixture was obtained.

After having brought the setpoint temperature to 140° C., the 2 copolymers and the antioxidant were introduced, with stirring, and the mixture was blended until a homogeneous mixture was obtained.

The petroleum jelly was then introduced with stirring at 140° C., in two steps, until a homogeneous mixture was obtained.

The mixture thus obtained was hot-cast at a temperature of about 120-130° C. onto an engraved flat plate forming the imprint of a square-mesh net or grid.

After cooling and demolding, the expected dressing was obtained in the form of a square-mesh net having a thickness of approximately 600 μm, a mesh size of about 2 mm and a grammage of about 450 g/m².

The dressings thus produced were placed between two temporary protective films made of silicone-treated polyester, 50 μm thick.

A self-supported interface dressing which has excellent cohesion and extensibility properties, but especially which does not pose any problem when it is handled with latex gloves since it does not “adhere”, was thus obtained.

1-11. (canceled)

12. A composition which has a low affinity with respect to latex gloves, comprising a hydrophobic matrix, wherein said hydrophobic matrix comprises:
for 100 parts by weight of a mixture P of a first styrene-saturated olefin-styrene triblock copolymer which has a viscosity of between 0.01 and 1 Pa·s as measured in a 5% (weight/weight) solution in toluene and a second styrene-saturated olefin-styrene triblock copolymer which has a viscosity of between 0.01 and 0.5 Pa·s as measured in a 15% (weight/weight) solution in toluene;
from 300 to 1000 parts by weight of a plasticizer H; and
from 90 to 600 parts by weight of petroleum jelly V;
it further being specified that:
the total amount, represented by P+H+V, of the mixture of copolymers, of plasticizer and of petroleum jelly is between 490 and 1700 parts by weight;
the ratio between the total amount of the mixture of copolymers, of plasticizer and of petroleum jelly and the amount of petroleum jelly, represented by P+H+V/V, is less than 11;
and said mixture P of 2 copolymers comprises at least 20% by weight of the first copolymer.

13. The composition as claimed in claim 12, wherein the hydrophobic matrix comprises:
for 100 parts by weight of a mixture P of a first styrene-saturated olefin-styrene triblock copolymer which has a viscosity of between 0.01 and 1 Pa·s as measured in a 5% (weight/weight) solution in toluene and a second styrene-saturated olefin-styrene triblock copolymer which has a viscosity of between 0.01 and 0.5 Pa·s as measured in a 15% (weight/weight) solution in toluene;
from 500 to 1000 parts by weight of a plasticizer H; and
from 100 to 400 parts by weight of petroleum jelly V;
it further being specified that:
the total amount, represented by P+H+V, of the mixture of copolymers, of plasticizer and of petroleum jelly is between 700 and 1200 parts by weight;
the ratio between the total amount of the mixture of copolymers, of plasticizer and of petroleum jelly and the amount of petroleum jelly, represented by P+H+V/V, is less than 8;
and said mixture P of 2 copolymers comprises at least 20% by weight of the first copolymer.

14. The composition as claimed in claim 12, wherein the plasticizer H is a plasticizing oil.

15. The composition as claimed in claim 12, wherein the mixture of 2 copolymers comprises at least 50% of the first copolymer which has a viscosity of between 0.01 and 1 Pa·s.

16. The composition as claimed in claim 12, wherein the mixture of 2 copolymers consists of 70% by weight of the first copolymer which has a viscosity of between 0.01 and 1 Pa·s and of 30% by weight of the second copolymer which has a viscosity of between 0.01 and 0.5 Pa·s.

17. The composition as claimed in claim 12, wherein the mixture of 2 copolymers consists of 70% by weight of the first copolymer which has a viscosity of between 0.01 and 1 Pa·s and of 30% by weight of the second copolymer which has a viscosity of between 0.01 and 0.5 Pa·s and wherein the hydrophobic matrix further comprises hydrocolloid particles in an amount of less than or equal to 25% by weight, relative to the total weight of said hydrophobic matrix.

18. The composition as claimed in claim 12, wherein the mixture of 2 copolymers consists of 70% by weight of the first copolymer which has a viscosity of between 0.01 and 1 Pa·s and of 30% by weight of the second copolymer which has a viscosity of between 0.01 and 0.5 Pa·s and wherein the hydrophobic matrix further comprises hydrocolloid particles in an amount of between 2% and 20% by weight, relative to the total weight of said hydrophobic matrix.

19. The composition as claimed in claim 12, wherein the mixture of 2 copolymers consists of 70% by weight of the first copolymer which has a viscosity of between 0.01 and 1 Pa·s and of 30% by weight of the second copolymer which has a viscosity of between 0.01 and 0.5 Pa·s and wherein the hydrophobic matrix further comprises hydrocolloid particles in an amount of between 10% and 15% by weight, relative to the total weight of said hydrophobic matrix.

20. The composition as claimed in claim 12, which further comprises one or more substances selected from the group consisting of substances which have a favorable role in wound treatment, bactericidal agents, bacteriostatic agents, painkillers, local anesthetics and anti-inflammatory agents, in an amount of between 0.01% and 20% by weight, relative to the total weight of the composition.

21. The composition as claimed in claim 12, wherein the mixture of 2 copolymers consists of 70% by weight of the first copolymer which has a viscosity of between 0.01 and 1 Pa·s and of 30% by weight of the second copolymer which has a viscosity of between 0.01 and 0.5 Pa·s and wherein the hydrophobic matrix further comprises hydrocolloid particles in an amount of between 2% and 20% by weight, relative to the total weight of said hydrophobic matrix; wherein said composition further comprises one or more substances selected from the group consisting of substances which have a favorable role in wound treatment, bactericidal agents, bacteriostatic agents, painkillers, local anesthetics and anti-inflammatory agents, in an amount of between 0.01% and 20% by weight, relative to the total weight of the composition.

22. The composition as claimed in claim 12, wherein the mixture of 2 copolymers consists of 70% by weight of the first copolymer which has a viscosity of between 0.01 and 1 Pa·s and of 30% by weight of the second copolymer which has a viscosity of between 0.01 and 0.5 Pa·s and wherein the hydrophobic matrix further comprises hydrocolloid particles in an amount of between 2% and 20% by weight, relative to the total weight of said hydrophobic matrix; wherein said composition further comprises one or more substances selected from the group consisting of substances which have a favorable role in wound treatment, bactericidal agents, bacteriostatic agents, painkillers, local anesthetics and anti-inflammatory agents, in an amount of between 1% and 15% by weight, relative to the total weight of the composition.

23. An interface dressing with reinforcement, with a support or which is self-supported, which comprises a composition as claimed in claim 12.

24. An interface dressing with reinforcement, with a support or which is self-supported as claimed in claim 23, wherein the mixture of 2 copolymers consists of 70% by weight of the first copolymer which has a viscosity of between 0.01 and 1 Pa·s and of 30% by weight of the second copolymer which has a viscosity of between 0.01 and 0.5 Pa·s and wherein the hydrophobic matrix further comprises hydrocolloid particles in an amount of between 2% and 20% by weight, relative to the total weight of said hydrophobic matrix; wherein said composition further comprises one or more substances selected from the group consisting of substances which have a favorable role in wound treatment, bactericidal agents, bacteriostatic agents, painkillers, local anesthetics and anti-inflammatory agents, in an amount of between 1% and 15% by weight, relative to the total weight of the composition.

25. A self-supported interface dressing formed from a thin layer of a composition comprising a hydrophobic matrix and comprising through-holes, wherein said hydrophobic matrix comprises:
for 100 parts by weight of a mixture P of a first styrene-saturated olefin-styrene triblock copolymer which has a viscosity of between 0.01 and 1 Pa·s as measured in a 5% (weight/weight) solution in toluene and a second styrene-saturated olefin-styrene triblock copolymer which has a viscosity of between 0.01 and 0.5 Pa·s measured in a 15% (weight/weight) solution in toluene;
from 500 to 1000 parts by weight of a plasticizer H; and
from 100 to 400 parts by weight of petroleum jelly V;
it further being specified that:
the total amount, represented by P+H+V, of the mixture of copolymers, of plasticizer and of petroleum jelly is between 700 and 1200 parts by weight;
the ratio between the total amount of the mixture of copolymers, of plasticizer and of petroleum jelly and the amount of petroleum jelly, represented by P+H+V/V, is less than 8;
and said mixture P of 2 copolymers comprises at last 30% by weight of the first copolymer.

26. A self-supported interface dressing as claimed in claim 25, wherein the plasticizer H is a plasticizing oil.

27. The self-supported interface dressing as claimed in claim 25, wherein the hydrophobic matrix further comprises hydrocolloid particles in an amount of less than or equal to 25% by weight, relative to the total weight of said hydrophobic matrix.

28. The self-supported interface dressing as claimed in claim 25, wherein the hydrophobic matrix comprises:
for 100 parts by weight of a mixture P of 2 styrene-saturated olefin-styrene triblock copolymers, a first which has a viscosity of between 0.01 and 1 Pa·s as measured in a 5% (weight/weight) solution in toluene and a second which has a viscosity of between 0.01 and 0.5 Pa·s as measured in a 15% (weight/weight) solution in toluene;
from 650 to 800 parts by weight of a plasticizer H; and
from 150 to 200 parts by weight of petroleum jelly V;
it further being specified that:
the total amount, represented by P+H+V, of the mixture of copolymers, of plasticizer and of petroleum jelly is between 900 and 1100 parts by weight;
the ratio between the total amount of the mixture of copolymers, of plasticizer and of petroleum jelly and the amount of petroleum jelly, represented by P+H+V/V, is less than 6;
said mixture P of 2 copolymers comprises at least 50% by weight of the first polymer;
and hydrocolloid particles in an amount of between 10% and 20% by weight relative to the total weight of the hydrophobic matrix.

29. The self-supported interface dressing as claimed in claim 25, wherein it is in the form of a breathable net of which the mesh size is about 2 mm, the thickness is about 600 microns and the grammage is about 450 g/m².
